(19) United States
(12) Patent Application Publication
(10) Pub. No.: US 2009/0287180 A1
(43) Pub. Date: Nov. 19, 2009

(54) DISPOSABLE PUMP RESERVOIR AND RELATED METHODS

(76) Inventor: Paul M. DiPerna, San Clemente, CA (US)

Correspondence Address: Luce, Forward, Hamilton & Scripps LLP, 2050 Main Street, Suite 600, Irvine, CA, 92614, US

(21) Appl. No.: 12/468,795

(22) Filed: May 19, 2009

Related U.S. Application Data
(60) Provisional application No. 61/054,420, filed on May 19, 2008.

(51) Int. Cl. A61M 5/168; G01F 1/00

(52) U.S. Cl. 604/500; 604/67; 702/45

(57) ABSTRACT

A device comprises a reusable and a disposable comprising at least one first chamber holding a flow material and a second chamber holding a gas. The disposable is removable from the reusable and maintains sterility when removed from the reusable. Flow metering device provide safety and allow for variably stroke frequency thereby modulating flow rate.

### RELATED APPLICATION

This application claims the benefit of and priority to U.S. Utility application Ser. Nos. 12/393,973, filed Feb. 26, 2009; 12/108,462, filed May 13, 2008; 12/020,498, filed Jan. 25, 2008; 11/744,819, filed May 4, 2007; 11/343,817, filed Jan. 31, 2006; and 61/054,420, filed May 19, 2008; the contents of which are incorporated by reference herein in their entirety.

### BACKGROUND

This disclosure relates to disposable pump reservoirs that are used in pumps, particularly infusion pumps.

### SUMMARY

A device comprises a reusable and a disposable comprising at least one first chamber for holding a gas and at least one second chamber for holding a flow material. The disposable assembly is removable from the reusable and maintains sterility when removed from the reusable.

According to a feature of the present disclosure, a device is disclosed comprising a reusable having at least one pressure sensor; a disposable comprising at least one first chamber and at least one second chamber holding a pressurized gas; and a slideable metering device. The disposable is removable from the reusable and maintains sterility when removed from the reusable and wherein when the disposable is interconnected with the reusable, the at least one second chamber is in fluid communication with the sensors.

According to a feature of the present disclosure, a device is disclosed comprising a reusable having at least one pressure sensor; and a disposable comprising at least one first chamber holding a flow material , a second chamber, and a third chamber holding a pressurized gas. The first chamber and second chamber are configured such that when the pressure in the second chamber increases, the volume of the second chamber increases and the volume of the third chamber decreases proportionally. The disposable is removable from the reusable and maintains sterility when removed from the reusable and wherein when the disposable is interconnected with the reusable, the at least one second chamber is in fluid communication with the sensors.

According to a feature of the present disclosure, a method is disclosed comprising providing an infusion pump having a reusable having at least one pressure sensor; and

a disposable comprising at least one first chamber and at least one second chamber holding a pressurized gas; and a slideable metering device. The disposable is removable from the reusable and maintains sterility when removed from the reusable and wherein when the disposable is interconnected with the reusable, the at least one second chamber is in fluid communication with the sensors.

### DRAWINGS

The above-mentioned features and objects of the present disclosure will become more apparent with reference to the following description taken in conjunction with the accompanying drawings wherein like reference numerals denote like elements and in which:

FIG. 1 is a perspective diagram of embodiments of the disposables of the present disclosure;

FIG. 2 is a perspective diagram of embodiments of the disposables of the present disclosure;

FIG. 3 is a perspective diagram of embodiments of disposable pump devices of the present disclosure;

FIG. 4 is a perspective diagram of embodiments of disposable pump devices of the present disclosure;

FIG. 5 is a perspective diagram of embodiments of the disposable assemblies of the present disclosure;

FIG. 6 is a perspective diagram of embodiments of the disposable assemblies of the present disclosure;

FIG. 7 is a perspective diagram of embodiments of the disposable assemblies of the present disclosure;

FIG. 8 is a perspective diagram of embodiments of the disposable assemblies of the present disclosure;

FIG. 9 is a perspective diagram of embodiments of the disposable assemblies of the present disclosure;

FIG. 10 is a perspective diagram of embodiments of the disposable assemblies of the present disclosure;

FIG. 11 is a cross-sectional diagram of embodiments of the valve assemblies of the present disclosure;

FIG. 12 is a cross-sectional diagram of embodiments of the valve assemblies of the present disclosure;

FIG. 13 is a perspective diagram of alternative embodiments of the disposable assemblies of the present disclosure; and

FIG. 14 is a perspective diagram of alternative embodiments of the disposable assemblies of the present disclosure.

### DETAILED DESCRIPTION

In the following detailed description of embodiments of the present disclosure, reference is made to the accompanying drawings in which like references indicate similar elements, and in which is shown by way of illustration specific embodiments in which the present disclosure may be practiced. These embodiments are described in sufficient detail to enable those skilled in the art to practice the present disclosure, and it is to be understood that other embodiments may be utilized and that logical, mechanical, electrical, functional, and other changes may be made without departing from the scope of the present disclosure. The following detailed description is, therefore, not to be taken in a limiting sense, and the scope of the present disclosure is defined only by the appended claims. As used in the present disclosure, the term “or ” shall be understood to be defined as a logical disjunction and shall not indicate an exclusive disjunction unless expressly indicated as such or notated as “xor.”

As used herein, the term “fluid” shall be understood to mean both a gas and a liquid.

As used herein, the term “real time” shall be understood to mean the instantaneous moment of an event/condition or the instantaneous moment of an event/condition plus short period of elapsed time used to make relevant measurements, optional computations, and communicate the measurement or computation, wherein the state of an event/condition being measured is substantially the same as that of the instantaneous moment irrespective of the elapsed time interval. Used in this context “substantially the same” shall be understood to mean that the data for the event/condition remains useful for the purpose for which it is being gathered after the elapsed time period.

Drug delivery devices such as infusion pumps are used to infuse medications or other biologically active substances into human or animal subjects. As used herein, the term “biologically active substance” means all types of medical and biological fluid used in the treatment of humans and animals including but not limited to peptides (such as insulin), analgesics, antiarrhythmics, steroids, hormones, nicotine, vitamins, anti-migraine medicine, anti-coagulants, local anesthetics, vaccines, allergens, muscle relaxants, and the like. It should also be recognized that the apparatus is suited for the delivery of fluid into mammals, plants, fish, reptiles, and birds. The dosage levels are typically small and must be maintained over long periods of time in order to sustain a desired effect or result in the subject. A typical application is the administration of pharmaceutical preparations, where the treatment is vital for correct biological activity. The dosage delivery in such instances is often critical, and effective feedback in the form of measured flow rates is seldom available with currently used devices.

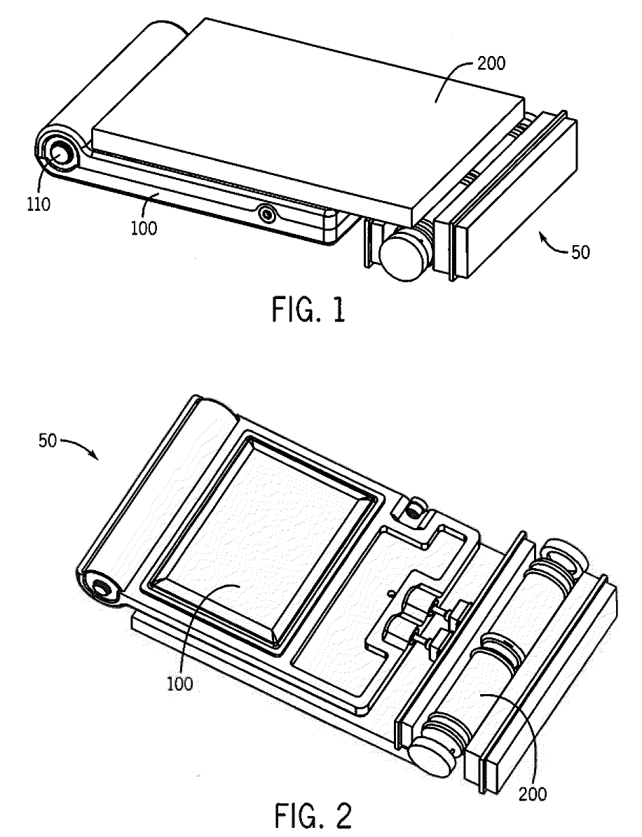

As illustrated according to embodiments in FIG. 1, disposable 100 containing at least one flow material reservoir for a pump is disclosed. Disposable 100 comprises a modular device that allows for the rapid exchange of old disposable 100 for new disposable 100 that is fully charged with flow material, according to embodiments. Moreover, the disposables 100 are disposed for exchange between devices while maintaining sterility. According to other embodiments, each sterile disposable 100 may be empty and filling is permitted by the end user.

As illustrated in FIGS. 1 and 2, infusion pump 50 is illustrated. Pump 50 may be any infusion pump, for example those devices that are incorporated by reference herein. Pump 50 comprises two major components, disposable 100 and reusable 200. Disposable 100, according to embodiments, comprises as least one first chamber 102 (flow material reservoir) holding a flow material. According to embodiments, disposable 100 may have more than one first chamber 102 for holding flow material. According to embodiments, first chamber 102 holding the flow material may vary in size depending on the needed volume of flow material.

According to embodiments, disposable 100 further comprises at least one battery 110 to power infusion pump 50, alleviating the need for battery lo to be built into reusable 200 and addressing issues related to battery life (because the battery is replaced each time disposable 100 is replaced). Finally, disposable comprises second chamber 104 (gas chamber) having a pressurized gas. The operation of such infusion pumps are described generally as incorporated by reference.

According to embodiments, reusable 200 comprises the electrical hardware, and in some cases sensors, for the computations necessary to calculate the flow rate or flowed volume of the flow material in real time. Moreover, reusable 200 comprises connectors capable of bringing the gas chamber(s) into gaseous communication with second chamber 104 in reusable 200, which according to embodiments may comprise a conduit, having sensors necessary for calculation of the dispensed volume of flow material. According to embodiments, the sensors may be pressure transducers. According to other embodiments, the sensors comprise acoustic volume measurement technology, for example as disclosed in U.S. Pat. Nos. 5,575,310; 5,755,683; and U.S. Patent Pub. No. 2007/0219496 which are incorporated by reference. Other sensors and sensing techniques are similarly contemplated, including: Doppler-based methods, Hall-effect sensors in combination with a vane or flapper valve; strain beams (e.g., related to flexible members over a fluid chamber to sense deflection of the flexible members); capacitance sensing plates, or thermal time of flight methods.

According to embodiments, reusable 200 also contains input and output devices, such as buttons, wheels, touch pads, touch screens, wireless connection devices, such as devices using Bluetooth (IEEE 802.15) or IEEE 802.11 wireless communication devices, and others that would be apparent to persons of ordinary skill in the art. These allow users to interact with the device and generally allow for users to communicate data from the devices of the present disclosure as desirable.

Because second chamber 104 of the reusable 200 and the disposable 100 are not sterile, reusables 200 can be exchanged with other reusables 200 of the same or different design configurations without breaking sterility because the points of contact between reusable 200 and disposable 100 are not sterile components.

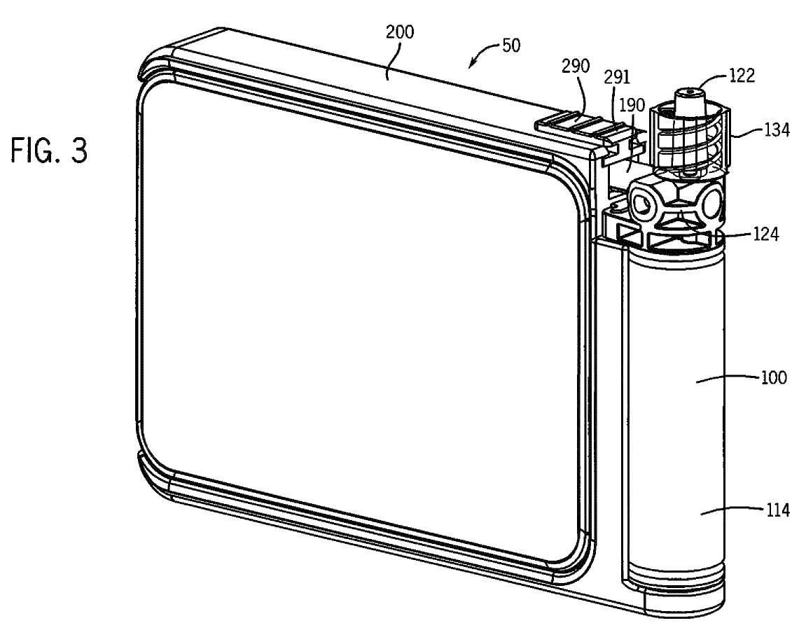

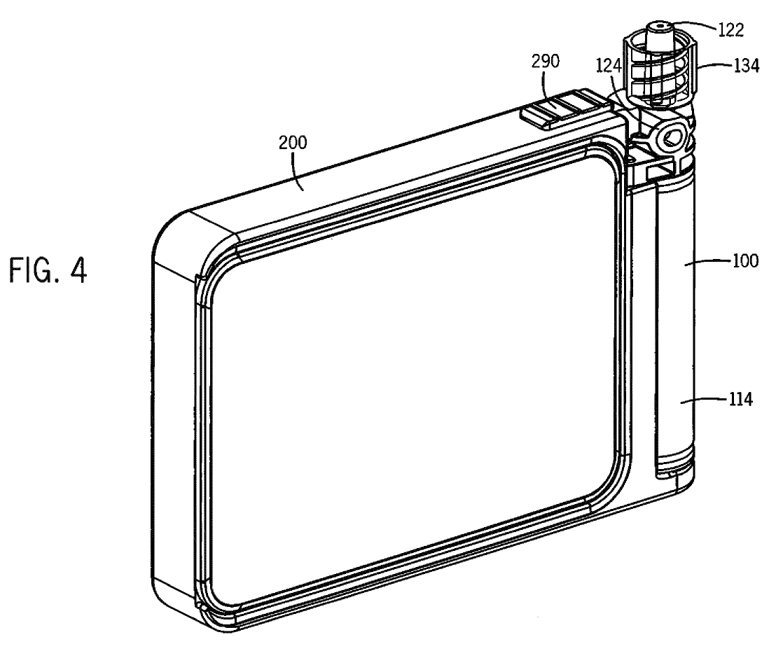

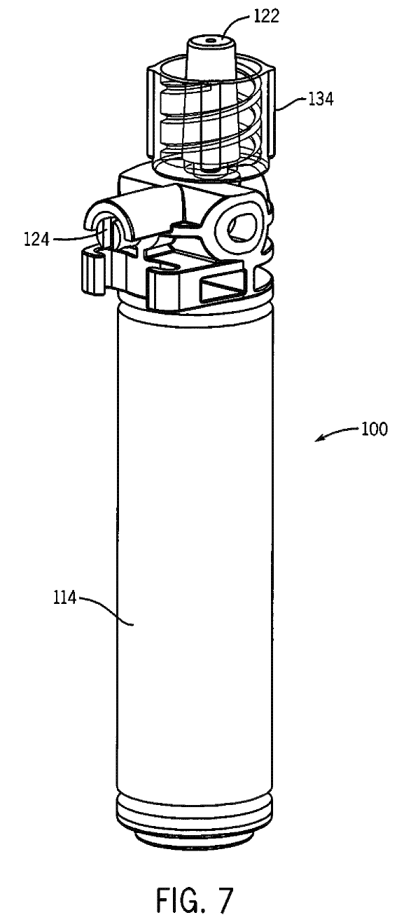

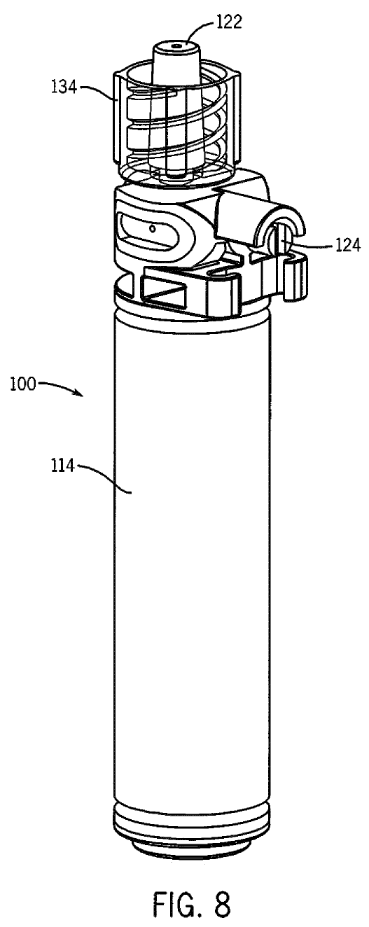

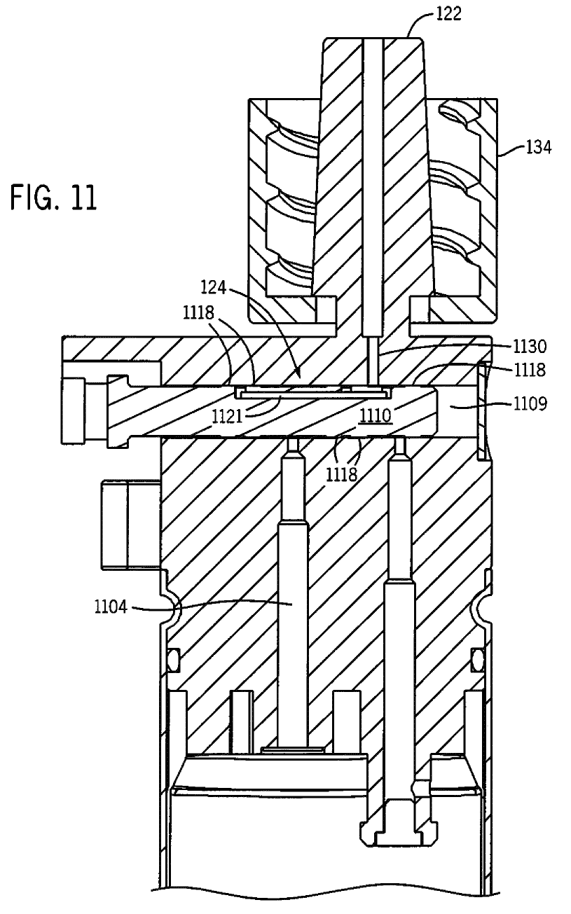

FIGS. 3-12 illustrate an embodiment of a disposable 100 and reusable 200. FIGS. 3 and 4 illustrate the embodiment where disposable 100 and reusable 200 are interconnected and working in conjunction with each other. According to embodiments, infusion pump assembly 250 comprises disposable 100 and reusable 200. Disposable 100 has housing 114 and flow metering device 124. According to embodiments, flow metering device 124 is a slideable metering device as disclosed in incorporated by reference U.S. Utility patent application Ser. No. 12/393,973, filed Feb. 26, 2009; in the instant drawings, lead lines with numbers in the 1000's correspond to structural members illustrated in the drawings of that patent application in the 100's (i.e., lead lines 1118 in the present disclosure corresponds with lead lines 118 in the application Ser. No. 12/393,973, with the corresponding description in the specification).

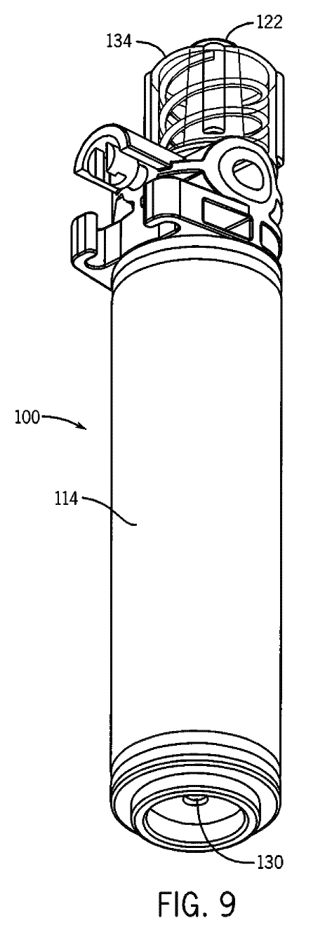

Disposable 100 and reusable 200 interconnect via a cannula-like device (not shown) that pierces second chamber septum 130 (FIG. 9) to put sensors disposed in reusable 200 in fluid communication with second chamber 104. Generally, cannula-like device is a device that is capable of sealably piercing second chamber septum 130 and allowing fluid (gas or liquid) to flow to the gas chamber in reusable 200 where the sensor(s) are housed. According to embodiments, sensors are pressure transducers, or the other sensors disclosed herein or incorporated by reference. Because placing reusable 200 into fluid communication with disposable 100 effects a loss of pressure in second chamber 104 as the fluid pressurizes the reusable gas chamber and the conduit between second chamber 104 and the reusable gas chamber, after inserting new disposable 100 into reusable 200, a calibration step may be performed, according to embodiments. According to other embodiments, however, no calibration step is necessary as the total volume of first chamber 102 is known and only changes in pressure in second chamber 104 pressure are measured. I.e., knowing the exact pressure of second chamber 104 is unnecessary to calculate the volume of flow material delivered in real time.

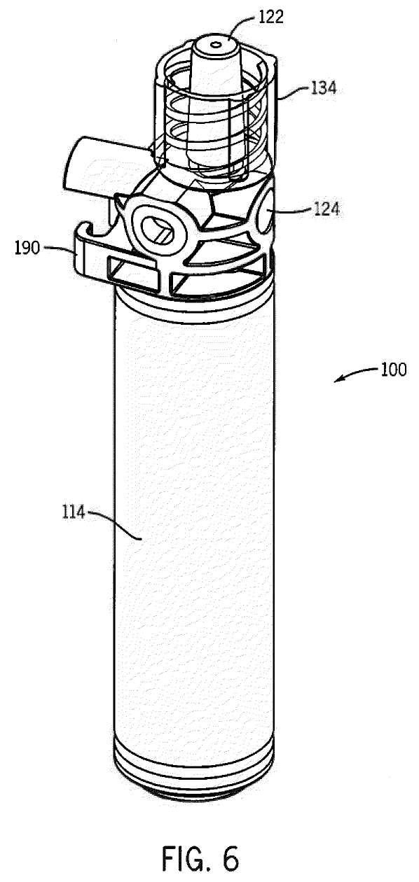

Disposable 100 also comprises securing member 190, which interconnects with securing device 290 in reusable 200. As illustrated in FIG. 6, securing member 190 is an L-shaped clasp. As it is inserted into securing device 290, securing member 190 displaces an interlocking member (not shown) as it passes by the interlocking member until it clears the interlocking member. Once clear, interlocking member returns to its original configuration, which interlocks with securing member 190, whereby interlocking member prevents securing member 190 from displacing interlocking member unless interlocking member is manually displaced by the user using switch 291. Artisans will understand the various devices that can be used to interconnect two members of a device as these are well known and understood generally.

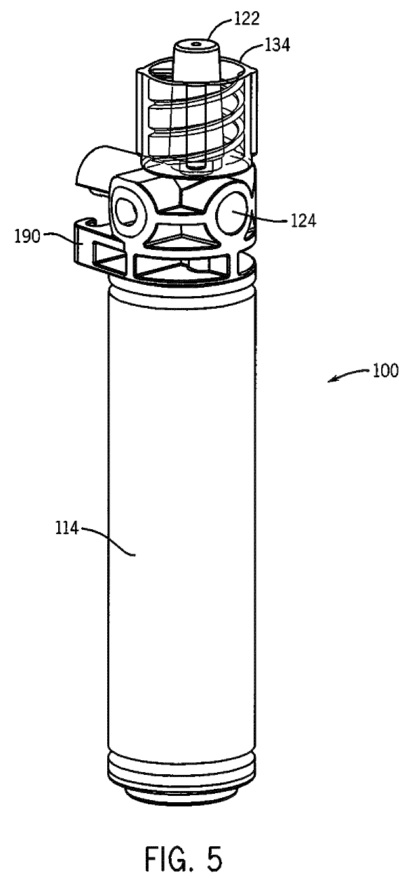

FIGS. 5-12 illustrate an embodiments of disposable 100 in more detail. More specifically, FIGS. 5-9 illustrate embodiments of disposable 100 in various exterior views. Housing 114 contains first chamber 102 and second chamber 104, as well as flow metering device 124. Together, the entire assembly is an infusion pump, wherein slideable metering device 124 is an optional, yet desirable component. Indeed, slideable flow metering device 124 couples the real-time feedback in flow rate and allows for the device to variably modulate flow rate. In other words, the device calculates exactly how much flow material is delivered with each aliquot. If it is determined that too much flow material was delivered in a unit of time, slideable flow metering device 124 will increase the frequency of each stroke, thereby delivering more flow material per unit time and visa versa when it is determined that not enough flow material has been delivered over a unit of time. Moreover, because slideable flow metering device 124 provides only a small aliquot of flow material at each stroke, it provides safety when used in conjunction with real time feedback of flow rate because at the most, only a small aliquot of flow material will be delivered, even in the most catastrophic of device failures.

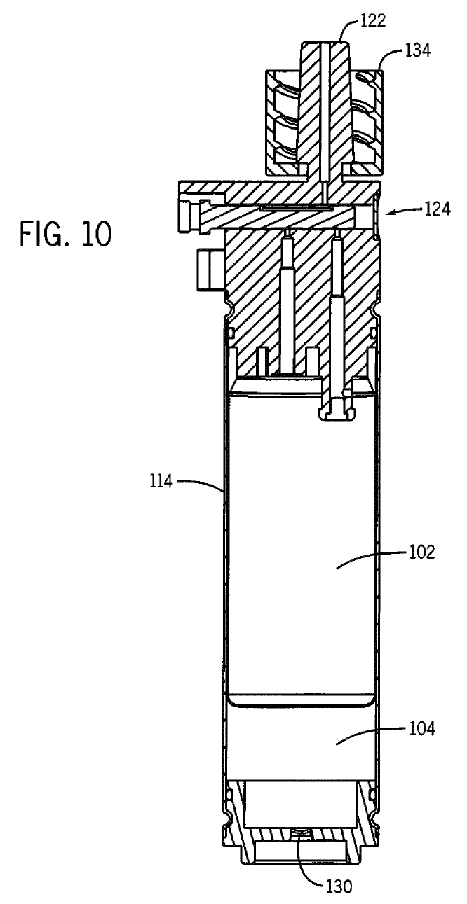

First chamber 102 holds the flow material. Second chamber 104 is a sealed, pressurized gas chamber. As flow material is permitted to escape first chamber 102, the pressure of the gas in second chamber 104 effects the flow by forcing the flow material to exit first chamber 102 via input conduit 1104, as shown in better detail in the cross sectional views of FIGS. 10-12). According to embodiments, operation of slideable flow metering device 124 is in accordance with the principles disclosed in U.S. Utility patent application Ser. No. 12/393,973, filed Feb. 26, 2009, which is incorporated by reference. (Note, lead lines in the 1000's in the instant drawings correspond to the application Ser. No. 12/393,973 lead lines in the 100's, with the appropriate description.)

Generally, actuation shaft 1110 having shaft channel 1121 resides in cavity 109. Movable seals 1118 define a series of sealed spaces. Flow material enters into a sealed space and files a chamber having compressible member 1138. When actuation shaft 1110 moves, flow material is dispensed from the chamber having compressible member 1138 through output conduit 1130. Output conduit 1130 is in fluid communication with output device 122, which comprises a connector, for example Leur connector 134, which are well known and understood in the art.

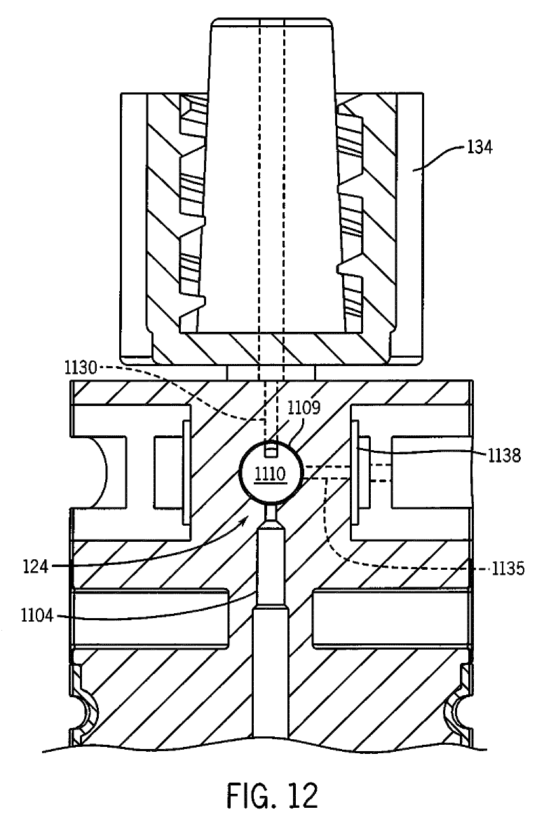

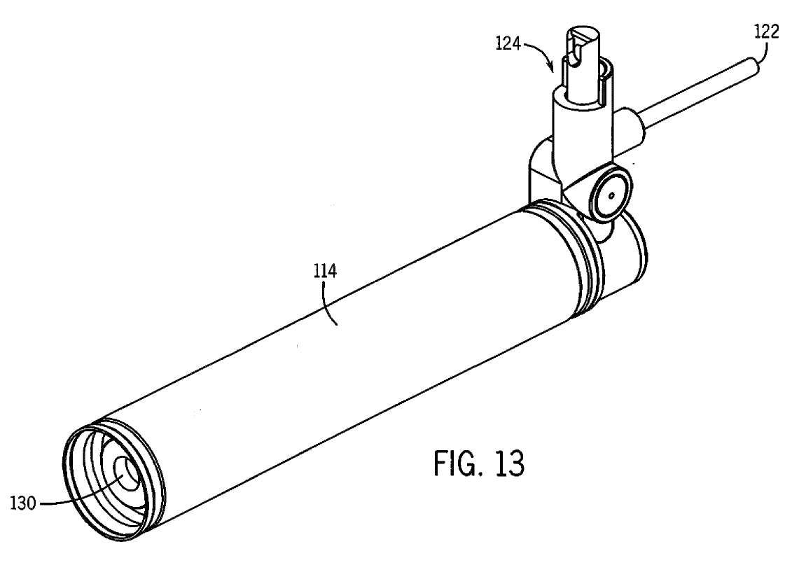

FIG. 12, shows a side cross sectional view, which better illustrates the system of conduits, including input conduit 1104, output conduit 1130, and chamber conduit 1135. According to embodiments, additional chambers having compressible members 1138 and chamber conduits 1135 may be disposed in the device to provide for delivery of different aliquot sizes, as disclosed herein or by incorporation.

According to other embodiments, a third chamber (not shown) having a pressurized gas releases small aliquots of pressurized gas into second chamber 104, thereby increasing the pressure in second chamber 104, which then causes flow of flow material from first chamber 102. In operation, many different variations of the possible infusion pumps disclosed are described in (including methods of operation and determination of real-time flow volume) U.S. Utility application Ser. Nos. 12/108,462, filed May 13, 2008; 12/020,498, filed Jan. 25, 2008; 11/744,819, filed May 4, 2007; 11/343,817, filed Jan. 31, 2006, now issued U.S. Pat. No. 7,374,556, which are incorporated by reference.

An alternative embodiment of a disposable assembly 600 is shown in FIG. 13 where the slideable flow metering device 124 is disposed in a different configuration relative to housing 114 and output device 122.

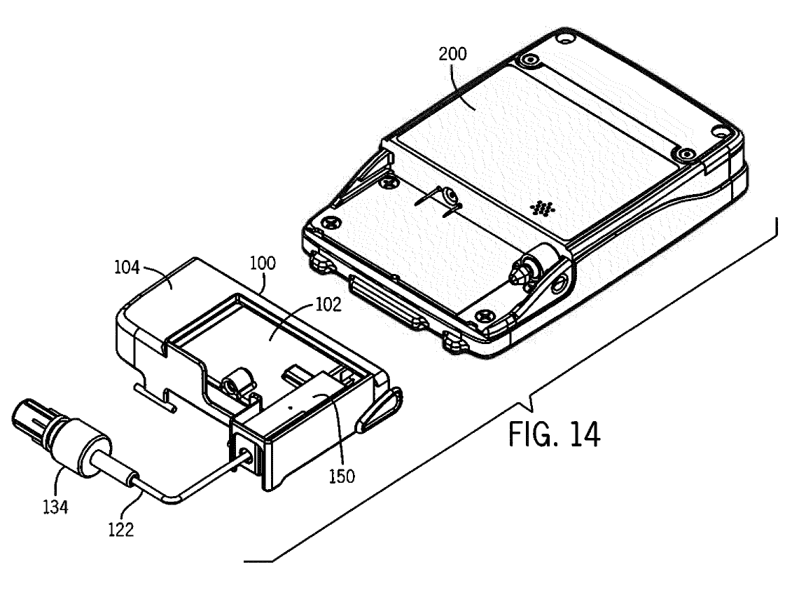

An alternative embodiment is shown in FIG. 14. Disposable 100 includes first chamber 102 that holds the flow material and second chamber 104 that holds a pressurized gas. The disposable 100 further comprises a valve assembly 150, which meters the flow of flow material for first chamber 102. Because the volume of flow material delivered from first chamber 102 can be calculated in about real time, valve assembly 150 therefore controls the rate at which flow material is delivered by opening and closing to effect flow or arrest of flow material. When valve assembly 150 is actuated, pressurized gas causes first chamber 102 to decrease in volume, thereby expelling flow material through valve assembly 150. Reusable 200 is configured to securely, but reversibly, house disposable 100. Moreover, reusable 200 comprises cannula-like device (not shown) that sealably pierces a septum separating second chamber 104 from the exterior environment, thereby putting sensors in fluid communication with second chamber 104 for determining volumes of second chamber 104 and therefore first chamber 102.

According to embodiments, disposable 100 is disposed to receive standard size disposable batteries, such as AA or AAA alkaline, nickel metal hydride, or lithium ion batteries. According to other embodiments, a proprietary sized battery 110 maybe used to conserve space or accommodate design constraints.

According to embodiments, disposable 100 may contain a solid state memory-type device, such as flash memory to log data and allow continuity of the data when disposable 100 is moved between devices.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A device comprising:
a reusable having at least one pressure sensor;
a disposable comprising at least one first chamber and at least one second chamber holding a pressurized gas; and
a slideable metering device;
wherein the disposable is removable from the reusable and maintains sterility when removed from the reusable and wherein when the disposable is interconnected with the reusable, the at least one second chamber is in fluid communication with the sensors.

2. The device of claim 1, wherein the slideable metering device comprises a plurality of chambers having a compressible member for receiving aliquots of flow material.

3. The device of claim 1, wherein the disposable assembly is disposed to receive disposable batteries.

4. The device of claim 1, wherein the device arrests flow when the disposable is disconnected from the reusable.

5. The device of claim 1, wherein the first chamber comprises a bag.

6. The device of claim 1, wherein the flow rate of the flow material is adjustable by changing the frequency of each stroke of the slideable metering device.

7. The device of claim 1, further comprising hardware on the reusable that calculates the flow rate of the flow material by determine the change in volume of the first chamber over a period of time;
wherein the data from the sensor is used to calculate the change in volume of the first chamber.

8. A device comprising:
a reusable having at least one pressure sensor; and
a disposable comprising at least one first chamber holding a flow material a second chamber, and a third chamber holding a pressurized gas; and
wherein the first chamber and second chamber are configured such that when the pressure in the second chamber increases, the volume of the second chamber increases and the volume of the third chamber decreases proportionally;
wherein the disposable is removable from the reusable and maintains sterility when removed from the reusable and wherein when the disposable is interconnected with the reusable, the at least one second chamber is in fluid communication with the sensors.

9. The device of claim 8, further comprising a valve assembly to communicate pressure between the first chamber and the second chamber.

10. A method comprising:
providing an infusion pump having a reusable having at least one pressure sensor;
a disposable comprising at least one first chamber and at least one second chamber holding a pressurized gas; and
a slideable metering device;
wherein the disposable is removable from the reusable and maintains sterility when removed from the reusable and wherein when the disposable is interconnected with the reusable, the at least one second chamber is in fluid communication with the sensors.

11. The method of claim 10, wherein the slideable metering device comprises a plurality of chambers having a compressible member for receiving aliquots of flow material.

12. The method of claim 10, wherein the disposable assembly is disposed to receive disposable batteries.

13. The method of claim 10, wherein the device arrests flow when the disposable is disconnected from the reusable.

14. The method of claim 10, where the first chamber comprises a bag.

15. The device of claim 10, wherein the flow rate of the flow material is adjustable by changing the frequency of each stroke of the slideable metering device.

16. The device of claim 10, further comprising hardware on the reusable that calculates the flow rate of the flow material by determine the change in volume of the first chamber over a period of time;
wherein the data from the sensor is used to calculate the change in volume of the first chamber.
